(19) United States
(12) Patent Application Publication
(10) Pub. No.: US 2016/0331735 A1
(43) Pub. Date: Nov. 17, 2016

(54) NOVEL FORMULATIONS FOR TREATMENT OF PAIN, NEUROPATHY, WOUNDS, AND ULCERS

(71) Applicant: Ashraf LATIF, South Orange, NJ (US)

(72) Inventor: Ashraf LATIF, South Orange, NJ (US)

(21) Appl. No.: 15/151,912

(22) Filed: May 11, 2016

Related U.S. Application Data
(60) Provisional application No. 62/159,700, filed on May 11, 2015.

(51) Int. Cl. A61K 31/4422; A61K 31/167; A61K 31/136; A61K 31/135; A61K 31/522; A61K 31/196; A61K 31/4168; A61K 31/195; A61K 31/192; A61K 31/4166; A61K 31/5575; A61K 31/4164; A61K 31/197; A61K 31/445; A61K 31/231; A61K 9/00

(52) U.S. Cl. CPC A61K 31/4422; A61K 9/0014; A61K 31/167; A61K 31/136; A61K 31/135; A61K 31/522; A61K 31/196; A61K 31/4168; A61K 31/195; A61K 31/192; A61K 31/4166; A61K 31/5575; A61K 31/4164; A61K 31/197; A61K 31/445; A61K 31/231

(57) ABSTRACT

The present disclosure describes various pharmaceutical formulations and compounds for transdermal treatment of pain, neuropathy, wounds and ulcers. The formulations may be particularly useful for treating pain, diabetic neuropathy or diabetic ulcers. In one embodiment a stock cream contains the following active ingredients: about 2 w-% Nifedipine and about 3-w-% Lidocaine, about 5-12 w-% Ketamine, about 2-4 w-% amitriptyline; about 8-10 w-% pentoxifylline, about 3-5 w-% mefenamic acid, and about 0.2-1 w-% clonidine. Other embodiments may exist and are contained under the purview of this disclosure.

### CLAIM OF PRIORITY

This application claims priority to U.S. application Ser. No. 62/159,700 which has a filing date of May 11, 2015, the contents of which are herein fully incorporated by reference in its entirety.

### FIELD OF THE EMBODIMENTS

The field of the embodiments of the present invention is related pharmaceutical formulations, more specifically, to compounded formulations for transdermal treatment of pain, neuropathy, wounds, and/or ulcers. The formulations are especially useful for the treatment of pain, diabetic neuropathy, and/or diabetic ulcers.

### BACKGROUND OF THE EMBODIMENTS

Topical or transdermal creams are known for delivering medication to or through the skin of a patient. There is constant need for new formulations of topical creams and ointments for various purposes and for patients with different needs and afflictions.

Diabetes is a metabolic condition that causes many complications for patient's suffering from the disease. For example, foot ulcers are a common complication of diabetes. These foot ulcers are most prevalent under the big toes as well as the balls of the feet. The ulcers form as a result of skin tissue breaking down and exposing underlying layers of tissue(s). The sores may affect the feet through the tissue and down to the bones. Such ulcers are very serious and ulcers are the most common reason for hospitalization of diabetics.

In addition to ulcers, diabetic patients are at risk of developing foot pain. Nerve damage is a long term effect of diabetes. Damaged nerves feel, at first, tingly and painful. The nerve damage may result in permanently reduced sensitivity to foot pain (neuropathy) and to painless wounds that can be further exacerbated by becoming ulcers.

Treatment of neuropathy, foot pain, and ulcers is of utmost importance because these symptoms, when untreated, can very easily lead to amputation of the affected appendage. The treatment depends highly on the cause of the symptom as well as on individual.

Topical ointments are available for diabetic pain, neuropathy, and ulcer treatments. However, not every formulation is suitable for every patient. Therefore there is a need for new formulations to address individual needs of various patients.

On the other hand, neuropathy ointments and creams are transdermal as opposed to other pain treatment creams and ointments. For example, creams for treating arthritis pain, while wound and scar creams typically do not have the transdermal or lipodermal function.

This disclosure solves the problem of providing individualized creams for various purposes, whether transdermal or lipodermal function is required or not. There are disclosures to various compounded ointments.

### Review of Related Technology:

U.S. Patent Application 2013/0085171 pertains to topically delivered medication for treatment of pain, inflammation, muscle fatigue, spasms, and/or other ailments. A transdermal cream may provide the effective topical administration of multiple medications simultaneously. The transdermal cream may include a salt load of approximately 30% or greater. The transdermal cream may include a unique base composition such that the transdermal cream may be able to remain stable and avoid degradation for six months or more and capable of effective delivery of active ingredient concentrations exceeding approximately 40% or more of the total formulation weight. The active ingredients may include a nerve depressant, NSAID, muscle relaxant, opiate agonist, local anesthetic, NMDA receptor antagonist, and a tricyclic antidepressant. In one embodiment, the transdermal cream may comprise ketamine HCL, gabapentin, clonidine HCL and baclofen. The transdermal cream may deliver an enhanced topical delivery flux of ketamine via a single transdermal application.

U.S. Patent Application 2004/0265364 pertains to compositions for transdermal pain relief. According to one embodiment, there is between 2% and 4% by weight amitriptyline, between 0.2% and 0.5% by weight clonidine, between 5% and 20% by weight Ketamine, between 2% and 6% by weight ketoprofen and optionally between 5% and 20% by weight Ketamine.

U.S. Patent Application 2004/0147534 pertains to a topical composition comprising about 6% to about 15% Nifedipine and about 6% to about 15% pentoxifylline for treating severe vascular occlusive wounds. The present invention also provides a method and a kit for treating the vascular occlusive wound by applying the composition to the open wound, and cleaning and dressing the wound at least once daily.

Various compounds are known in the art. However, their makeup and mechanism of action are substantially different from the present disclosure. The other inventions also fail to solve all the problems taught by the present disclosure. At least one embodiment of this invention is presented in the drawings below and will be described in more detail herein.

### SUMMARY OF THE EMBODIMENTS

The present invention and its embodiments relate to topically delivered compounded medications for treatment of pain, neuropathy, wounds, ulcers and scars.

In one embodiment of the present invention there is a topical stock formulation comprising an emollient cream; and Nifedipine:Lidocaine in ratio of about 2:3 or about 4:6.

In another embodiment of the present invention there is a method for making individualized topical creams for treating pain, neuropathy or ulcers, said method comprising the steps of: optionally diluting topical stock formulation with emollient cream, and mixing predetermined ingredients in the topical stock formulation or in the diluted stock formulation.

In general, the present invention succeeds in conferring the following, and others not mentioned, benefits and objectives.

In one aspect of the invention, there is provided compounded medication for topical administration of multiple medications simultaneously to treat one or more conditions.

In one aspect of the invention there is provided a method to individualize topical formulations for treating at least pain, neuropathy pain, wounds, ulcers and scars.

In one aspect of the invention there is a stock formulation is provided for a starting material of individualizing topical ointments for patients.

In one aspect of the invention there is a stock formulation is provided comprising an emollient cream, Lidocaine and Nifedipine.

In one aspect of the invention there is provided compounded topical formulations for treating diabetic neuropathy pain or diabetic ulcers and scars.

In one aspect of the invention topical formulations for treating pain are provided, said formulations comprising mixtures of: Ketamine, Diclofenac, Baclofen, Bupivacaine, Gabapentin, Ibuprofen, cetyl myristoleate, Lidocaine and Nifedipine.

In one aspect of the invention topical formulations for treating neuropathy are provided, said formulations comprising mixtures of: Ketamine, amitriptyline, Nifedipine, pentoxifylline, mefenamic acid and clonidine.

In one aspect of the invention topical formulations for treating ulcers are provided, said formulations comprising mixtures of phenytoin, misoprostol, metronidazole, Nifedipine and Lidocaine.

### DETAILED DESCRIPTION OF THE EMBODIMENTS

The present invention provides topical creams for treatment of at least pain, treatment of neuropathy and treatment of wounds, ulcers and scars. The embodiments of the present invention are particularly useful for providing topical creams for treating at least diabetic neuropathy and diabetic foot ulcers, and scars.

The topical cream for treating pain may be used to treat arthritis pain, osteoarthritis, rheumatoid arthritis, or other arthritic conditions as well as foot pain of diabetics.

The topical cream for treating neuropathy is intended for diabetics but may also be used for neuropathy pain caused by other than diabetes.

The topical cream for treating ulcers and wounds is intended for diabetics but may also be used for healing any serious wounds.

In one embodiment of this invention the wound ointment may be supplemented by ingredients healing scars. In one embodiment the scar healing ointment may be provided separately.

The topical creams of this invention are preferably in emollient cream or hydrogel base. The topical creams for treating pain and neuropathy of this invention include compositions capable of passing through the skin of the subject (usually a mammalian subject, particularly a human being).

This invention provides a stock cream and a method to prepare the stock cream and methods to prepare individualized transdermal or non-transdermal creams from the stock cream as well as alternative formulations for the individualized creams.

The invention and its embodiments are now described by way of the following non-limiting examples.

### Example 1

### Preparation of Stock Formulation

A stock formulation may be prepared into an emollient cream or hydrogel base. The emollient base may be, for example, pentravan gel. The hydrogel base may also be a Spirawash® gel or equivalent.

Preferably, the stock formulation is prepared to have a Nifedipine:Lidocaine ratio of about 4:6 or about 2:3. Preferably, the Nifedipine concentration in the stock formulation is about 2 w-% or about 4 w-% and the Lidocaine concentration is about 3 w-% or about 6 w-%. The stock cream may be prepared several days before it is to be used to prepare the neuropathy or the ulcer cream or the like.

### Example 2

### Preparation of a Topical Cream to Treat Neuropathy

A topical cream for treating neuropathy may be made of the stock cream. In a preferred embodiment, a composition that allows the active ingredients to penetrate the skin is included into the stock cream. According to one preferred embodiment, a transdermal or lipodermal ingredient is added. According to one preferred embodiment, the stock cream may be made in a base comprising lecithin organogel. According to one preferred embodiment, the stock cream may be made in a base comprising phonogel. According to one preferred embodiment, the transdermal or lipodermal ingredient is a combination of lecithin organogel and phonogel.

The stock formulation may be diluted, if needed, with the emollient cream to achieve stock having about 2 w-% Nifedipine and about 3 w-% Lidocaine. The following active ingredients may be added to the stock cream: about 2 w-% Nifedipine, about 3-w-% Lidocaine, about 5-12 w-% Ketamine, about 2-4 w-% amitriptyline; about 8-10 w-% pentoxifylline, about 3-5 w-% mefenamic acid, and about 0.2-1 w-% clonidine or any combination thereof. Additionally the following ingredients may be included into the formulation: about 3-7 w-%

Gabapentin, about 3-7 w-% flurbiprofen. Optionally, about 3-6 w-% of Ibuprofen or ketoprofen may be included. According to one embodiment, the final formulation includes: about 10 w-% Ketamine, about 2 w-% Amitriptyline, about 2 w-% Nifedipine, about 8 w-% Pentoxifylline, about 3%-w Mefenamic acid, about 0.3 w-% Clonidine, and about 3 w-% Lidocaine. The cream is used about 3 to 4 times per day by applying about 2 grams of the cream on the skin.

### Example 3

### Preparation of a Topical Cream for Treatment of Wounds and Ulcers

A topical cream for treating wounds and ulcers may be made of the stock formulation. The stock formulation may be diluted with emollient cream so as to have the desired content of Nifedipine and Lidocaine.

Preferably, a stock formulation having about 4 w-% Nifedipine and about 6 w-% Lidocaine is used. The following ingredients may be mixed into the stock formulation: about 5-8 w-% Phenytoin; about 0.002-4 w-% Misoprostol; about 2-5 w-% metronidazole, or any combination thereof. Optionally, about 2-3 w-% of levocetirizine or about 1-2 w-% of fluticasone may be added to the mixture. According to one embodiment, the formulation contains: about 7 w-% phenytoin, about 4 w-% misoprostol, about 5 w-% metronidazole, about 4 w-% Nifedipine, and about 6 w-% Lidocaine, or any combination thereof. The topical cream is intended to be applied about twice a day on the wound.

### Example 4

### Preparation of a Topical Cream for Treatment of Pain

A topical cream for treating pain may be made of the stock cream. A composition that allows the active ingredients to penetrate the skin may be included into the stock formulation. According to one preferred embodiment, a transdermal or lipodermal ingredient is added. According to one preferred embodiment, the stock cream may be made in a base comprising lecithin organogel. According to one preferred embodiment, the stock cream may be made in a base comprising phonogel. According to one preferred embodiment, the transdermal or lipodermal ingredient is a combination of lecithin organogel and phonogel.

Preferably, a stock formulation having about 2 w-% Nifedipine and about 3 w-% Lidocaine is used. The following active ingredients are added to the stock formulation: about 5-12 w-% Ketamine; about 2-6 w-% Diclofenac; about 2-5 w-% Baclofen; about 0.5-2 w-% Bupivacaine, about 4-8 w-% Gabapentin, about 2-5 w-% Ibuprofen; about 2-7 w-% cetyl myristoleate, or any combination thereof. According to one embodiment, the final formulation includes: about 10 w-% Ketamine, about 5 w-% Diclofenac, about 3 w-% Baclofen, about 3 w-% Bupivacaine, about 6 w-% Gabapentin, about Ibuprofen 3 w-%, about 2 w-% Nifedipine, about 3 w-% Lidocaine, and about 5 w-% cetyl myristoleate, or any combination thereof. The cream is intended to be used about 3 to 4 times per day by applying about 2 grams of the cream on the skin.

### Example 5

### Preparation of a Topical Cream for Treatment Of Pain

A topical cream for treating pain may be made of the stock cream. A composition that allows the active ingredients to penetrate the skin may be included into the stock formulation. In other embodiments no stock cream is required. According to one preferred embodiment, a transdermal or lipodermal ingredient is added to the formulation. According to one preferred embodiment, the transdermal or lipodermal ingredient is a combination of lecithin organogel and phonogel.

In a preferred embodiment, a 3 w-% Diclofenac gel is mixed with a 5 w-% Lidocaine ointment. However, the Diclofenac gel may contain about 1 w-% to about 10 w-% Diclofenac and the ointment may contain about 1 w-% to about 15 w-% Lidocaine. It is preferable that regardless of the weight percentage utilized that the ratio of Diclofenac gel to Lidocaine ointment be about 100 g:144 g respectively.

In another preferred embodiment, there is a cream having about 1 w-% to about 5 w-% Diclofenac, about 2 w-% to about 10 w-% Gabapentin, and about 0.5 w-% to about 3.5 w-% Lidocaine. In a most preferred embodiment, the cream contains about 1.5 w-% Diclofenac, about 6 w-% Gabapentin, and about 2.25 w-% of Lidocaine.

### Example 6

### Preparation of a Topical Cream for Treatment Of Pain, Neuropathy, and Promotion of Circulation

A topical cream for treating pain may be made of the stock cream. A composition that allows the active ingredients to penetrate the skin may be included into the stock formulation. In other embodiments no stock cream is required. According to one preferred embodiment, a transdermal or lipodermal ingredient is added to the formulation. According to one preferred embodiment, the transdermal or lipodermal ingredient is a combination of lecithin organogel and phonogel.

In a preferred embodiment, there is a cream having about 1 w-% to about 5 w-% Diclofenac, about 2 w-% to about 10 w-% Gabapentin, about 0.5 w-% to about 3.5 w-% Lidocaine, and about 3 w-% to about 10 w-% Pentoxifylline. In a most preferred embodiment, the cream contains about 1.5 w-% Diclofenac, about 6 w-% Gabapentin, and about 2.25 w-% of Lidocaine, and about 5 w-% Pentoxifylline.

What is claimed is:

1. An individualized topical cream for treating pain, neuropathy or wounds and ulcers, said cream comprising Nifedipine and Lidocaine in an emollient stock formulation.

2. The topical cream of claim 1, wherein the cream comprises Nifedipine and Lidocaine in a ratio of about 2:3 (w/w).

3. The topical cram of claim 2, wherein Nifedipine concentration is about 2 w-% and Lidocaine concentration is about 3 w-%.

4. The topical cream of claim 2, wherein Nifedipine concentration is about 4 w-% and Lidocaine concentration is about 6 w-%.

5. The topical cream of claim 2, wherein the cream is individualized for treatment of neuropathy and additionally comprises Ketamine, amitriptyline, pentoxifylline, mefenamic acid and clonidine and a transdermal and lipodermal base ingredient.

6. The topical cream of claim 5, wherein the transdermal base ingredient lecithin organogel or phonogel or combination thereof.

7. The topical cream of claim 6, wherein the cream comprises about 5-12 w-% Ketamine, about 2-4 w-% amitriptyline, about 2 w-% Nifedipine, about 3 w-% Lidocaine, about 8-10 w-% pentoxifylline, about 3-5 w-% mefenamic acid, and about 0.2-1 w-% clonidine.

8. The topical cream of claim 7, wherein the cream comprises about 10 w-% Ketamine, about 2 w-% amitriptyline, about 2 w-% Nifedipine, about 3w-% Lidocaine, about 8 w-% pentoxifylline, about 3 w-% mefenamic acid, and about 0.3 w-% clonidine.

9. The topical cream of claim 7, wherein the cream additionally comprises about 3-7 w-% Gabapentin and about 3-7 w-% flurbiprofen.

10. The topical cream of claim 7, wherein the cream additionally comprises about 3-5 w-% Ibuprofen or ketoprofen.

11. The topical cream of claim 2, wherein the cream is individualized for treatment of wounds and ulcers and additionally comprises phenytoin, Misoprostol, and metronidazole.

12. The topical cream of claim 11, wherein the cream comprises about 5-8 w-% phenytoin, about 0.002-4 w-% misoprostol, about 2-5 w-% metronidazole, about 2 w-% Nifedipine and about 3 w-% Lidocaine.

13. The topical cream of claim 11, wherein the cream comprises about 2 w-% Nifedipine, about 3 w-% Lidocaine, about 8 w-% phenytoin, about 0.002 w-% misoprostol and about 5-w% of metronidazole.

14. The topical cream of claim 13 further comprising levocetirizine or fluticasone.

15. The topical cream of claim 14 comprising about 2% levocetirizine or about 1% fluticasone.

16. The topical cream of claim 2, wherein the cream is individualized for treatment of pain and additionally comprises Ketamine, Diclofenac, Baclofen, Bupivacaine, Gabapentin, Ibuprofen, cetyl myristoleate and a transdermal and lipodermal base ingredient.

17. The topical cream of claim 16, wherein the cream comprises about 5-12 w-% Ketamine, about 2-6 w-% Diclofenac, about 2-5 w-% Baclofen, about 0.5-2 w-% Bupivacaine, about 4-8 w-% Ibuprofen, about 2-7 w-% cetyl myristoleate, about 2 w-% Nifedipine and about 3 w-% Lidocaine.

18. The topical cream of claim 17, wherein the cream comprises about 10 w-% Ketamine, about 5 w-% Diclofenac, about 23 w-% Baclofen, about 12 w-% Bupivacaine, about 3 w-% Ibuprofen, about 5 w-% cetyl myristoleate, about 2 w-% Nifedipine and 3 w-% Lidocaine.

19. A method for making individualized topical creams for treating pain, neuropathy or ulcers, said method comprising the steps of:
a) optionally diluting topical stock formulation of claim 1 with emollient cream, and
b) mixing predetermined ingredients in the topical stock formulation of claim 1 or in the diluted stock of step a).

20. The method of claim 20, wherein the individualized topical cream is for treating neuropathy and the predetermined ingredients comprise Ketamine, amitriptyline, pentoxifylline, mefenamic acid, and clonidine and a transdermal and lipodermal base ingredient.

21. The method of claim 20, wherein the individualized topical cream is for treating pain and the predetermined ingredients comprise Ketamine, Diclofenac, Baclofen, Bupivacaine, Gabapentin, Ibuprofen, cetyl myristoleate, and at least one of a transdermal and lipodermal base ingredient.

22. The method of claim 20, wherein the individualized topical cream is for treating ulcers and the predetermined ingredients comprise phenytoin, Misoprostol, and metronidazole.

23. The method of claim 23, wherein the predetermined ingredients further comprise levocetirizine and fluticasone.

24. A topical cream for treating pain, neuropathy or wounds and ulcers, said topical cream comprising:
about 1 w-% to about 5 w-% Diclofenac;
about 2 w-% to about 10 w-% Gabapentin; and
about 0.5 w-% to about 3.5 w-% Lidocaine.

25. The cream of claim 24 further comprising:
about 3 w-% to about 10 w-% Pentoxifylline.

26. The cream of claim 25 wherein the cream contains about 1.5 w-% Diclofenac, about 6 w-% Gabapentin, and about 2.25 w-% of Lidocaine, and about 5 w-% Pentoxifylline.

27. The cream of claim 24 wherein the cream contains about 1.5 w-% Diclofenac, about 6 w-% Gabapentin, and about 2.25 w-% of Lidocaine.

28. A topical cream for treating pain, neuropathy or wounds and ulcers, said topical cream comprising:
about 3 w-% Diclofenac; and
about 5 w-% Lidocaine.
